(19) United States
(12) Patent Application Publication
(10) Pub. No.: US 2019/0298205 A1
(43) Pub. Date: Oct. 3, 2019

(54) SYSTEMS AND METHODS FOR PROVIDING VISUAL BIOFEEDBACK

(71) Applicant: Li Ge, San Francisco, CA (US)

(72) Inventor: Li Ge, San Francisco, CA (US)

(21) Appl. No.: 16/370,534

(22) Filed: Mar. 29, 2019

Related U.S. Application Data
(60) Provisional application No. 62/651,645, filed on Apr. 2, 2018.

(51) Int. Cl. A61B 5/04; A61B 5/00

(52) U.S. Cl. CPC A61B 5/04012; A61B 5/742; A61B 5/486; A61B 5/7285

(57) ABSTRACT

The present invention is embodied in systems, methods, and non-transitory computer-readable media configured to receive video data, and corresponding video timestamp data, of a subject performing functional movements; receive electromyography data, and corresponding electromyography timestamp data, from an electromyograph attached to the subject performing functional movements; synchronize the received electromyography data and the received video data using the received electromyography timestamp data and the received video timestamp data; generate at least one electromyogram from the synchronized electromyography data; and display the synchronized video data and the at least one electromyogram.

### CROSS REFERENCE TO RELATED APPLICATIONS

This application claims the benefit under 35 U.S.C. § 119(e) of U.S. Provisional Application Ser. No. 62/651,645, filed Apr. 2, 2018, the content of which is incorporated by reference in its entirety into the present disclosure.

### FIELD OF THE INVENTION

This invention relates generally to electromyography and, more particularly, to systems and methods of providing visual biofeedback comprising video and at least one electromyogram synchronized with the video through timestamp data.

### BACKGROUND

In biomechanics, proximal muscles stabilize proximal joints and assist distal muscles as they move around or along a distal joint to create a range of motion. The multifidus muscle, for example, is a proximal muscle that stabilizes the spine and functions together with transversus abdominis and pelvic floor muscles to stabilize the back and pelvis before movement of the arms or legs.

Experts have long advocated for strengthening proximal muscles to address musculoskeletal conditions caused by unstable joints. More recently, however, experts have begun to identify the importance of optimizing muscle-activation timing to improve biomechanics and prevent injuries caused by faulty movement patterns.

One challenge of applying this advice to real-life treatment or training regimens is that proximal muscles can be deep and small, and, therefore, difficult to observe. Traditionally, therapists and other movement professionals have had to maintain contact between their fingers and the proximal muscle of interest to determine whether the subject's muscle has been activated at a specific moment. When the subject's movement is quick or complex, movement specialists have had to rely on their bare-eye observations to guess the muscle firing patterns. As a result, the traditional methods are not objective, quantifiable, or reliable.

Electromyography (EMG) is used as an electrodiagnostic technique for evaluating and recording the electrical activity produced by skeletal muscles. EMG is performed using an instrument called an electromyograph to produce an electromyogram. The electromyograph detects the electric potential signals generated by muscle cells when the cells are electrically or neurologically activated. These signals can be processed to produce the electromyogram, which is a waveform that reflects the sum of the electrical contributions made by the active motor units in the vicinity of the electromyograph.

These limited functions all are satisfactory, but they fail to provide other valuable functions as well. For example, while EMG is widely used to detect and analyze muscle activation, existing methods have proven inadequate for optimizing muscle activation timing. This is because it can be difficult or impossible to determine from EMG data alone when the target muscle activated during the subject's functional movement, and whether the activation occurred before, for example, movement of a distal limb.

It should be appreciated that there is a need for an improved EMG method that provides muscle activation patterns of target muscles during complex, functional movement, while providing insight on the timing of the muscle activation relative to the functional movement. The present invention fulfills this need and provides further related advantages.

### BRIEF SUMMARY OF THE INVENTION

The present invention is embodied in systems, methods, and non-transitory computer-readable media configured to receive video data, and corresponding video timestamp data, of a subject performing functional movements; receive electromyography data, and corresponding electromyography timestamp data, from an electromyograph attached to the subject performing functional movements; synchronize the received electromyography data and the received video data using the received electromyography timestamp data and the received video timestamp data; generate at least one electromyogram from the synchronized electromyography data; and display the synchronized video data and the at least one electromyogram.

In one embodiment, the electromyograph can be a surface electromyograph. In another embodiment the electromyograph can comprise a first electromyograph, wherein the electromyography data and electromyography timestamp data received from the first electromyograph can comprise electromyography data representing electrical activity produced by at least one proximal muscle selected from a group consisting of: transverse abdominis, multifidus, diaphragm, pelvic floor, gluteus medius, gluteus minimis, lower trapezius, suprasinatus, infraspinatus, teres minor, and subscapularis.

In one embodiment, the electromyograph can further comprise a second electromyograph, wherein the electromyography data and electromyography timestamp data received from the second electromyograph can comprise electromyography data representing electrical activity produced by at least one distal muscle. In another embodiment, the electromyography data and electromyography timestamp data received from the second electromyograph can comprise electromyography data representing electrical activity produced by at least one distal muscle selected from a group consisting of: tensor fascia latae, serratus anterior, deltoid, erector spinae, and hamstring.

In one embodiment, receiving the electromyography data and the corresponding electromyography timestamp data can comprise receiving electromyography data and electromyography timestamp data from both the first and second electromyographs.

In one embodiment, the at least one proximal muscle is the gluteus medius and the at least one distal muscle is the tensor fascia latae. In another embodiment, the at least one proximal muscle is the suprasinatus, infraspinatus, teres minor, or subscapularis, and the at least one distal muscle is the upper trapezius or deltoid. In a further embodiment, the at least one proximal muscle is the multifidus and the at least one distal muscle is the erector spinae. In an additional embodiment, the at least one proximal muscle is the gluteus maximus and the at least one distal muscle is the hamstring.

In one embodiment, receiving the electromyography data and the corresponding electromyography timestamp data can further comprise aggregating the electromyography data and the electromyography timestamp data received from the first and second electromyographs.

In one embodiment, each of the first and second electromyographs can comprise a plurality of electrodes.

In one embodiment, displaying the synchronized video data and the at least one electromyogram can comprise adding the at least one electromyogram to the synchronized video data.

In one embodiment, the electromyography data, the electromyography timestamp, the video data, and the video timestamp data can be received by wireless communication.

In one embodiment, the systems, methods, and non-transitory computer-readable media can be further configured to synchronize the at least one electromyogram with an anatomy illustration; and display the synchronized anatomy animation and the at least one electromyogram.

The present invention is also embodied methods of strength training. In one embodiment, the method comprises activating a target proximal muscle and a distal muscle during a functional movement; receiving visual biofeedback; and adjusting muscle activation timing of the target proximal muscle during the functional movement in response to the visual feedback. The visual feedback can comprise video of the functional movement and at least one electromyogram.

In one embodiment, the electromyogram can be generated from electromyography data received from a first surface electromyograph and electromyography data received from a second surface electromyograph.

In one embodiment, the electromyography data received from the first surface electromyograph can comprise electromyography data representing electrical activity produced by the target proximal muscle.

In one embodiment, the electromyography data received from the second surface electromyograph can comprise electromyography data representing electrical activity produced by the distal muscle.

In one embodiment, the target proximal muscle is the gluteus medius and the distal muscle is the tensor fascia latae. In another embodiment, the target proximal muscle is the suprasinatus, infraspinatus, teres minor, or subscapularis, and the distal muscle is the upper trapezius or deltoid. In a further embodiment, the target proximal muscle is the multifidus and the distal muscle is the erector spinae. In an additional embodiment, the target proximal muscle is the gluteus maximus and the distal muscle is the hamstring.

In one embodiment, the video of the functional movement can be synchronized with the at least one electromyogram by synchronizing video timestamp data and electromyography timestamp data from the first and second surface electromyographs.

Each feature or concept outlined above is independent and can be combined with the other features or concepts outlined above or with any other feature or concept disclosed in this application. Other features and advantages of the invention should become apparent from the following description of the preferred embodiments, taken in conjunction with the accompanying drawings, which illustrate, by way of example, the principles of the invention.

### BRIEF DESCRIPTION OF THE DRAWINGS

FIG. 1 illustrates a system including a visual biofeedback module in accordance with one embodiment.

FIG. 2 illustrates a reception module in accordance with one embodiment.

FIG. 3 illustrates a display module in accordance with one embodiment.

FIG. 4 illustrates an example display scenario in accordance with one embodiment.

FIG. 5 illustrates an example method associated with displaying video biofeedback comprising video and at least one electromyogram synchronized with the video in accordance with one embodiment.

FIG. 6 illustrates a network diagram of an example system that can be used in various scenarios in accordance with one embodiment.

FIG. 7 illustrates an example of a computer system or computing device that can be used in various scenarios in accordance with one embodiment.

### DETAILED DESCRIPTION OF THE PREFERRED EMBODIMENTS

An improved approach rooted in computer technology overcomes the foregoing and other disadvantages associated with conventional methods. In general, a visual biofeedback module can receive EMG data collected as a subject performs functional movements, along with corresponding video data of the subject performing the functional movements. The visual biofeedback module can then synchronize the received EMG data and the received video data using timestamp data. Once the video and EMG data are synchronized, the visual biofeedback module can generate an electromyogram from the EMG data and display the synchronized video data together with the electromyogram. In this way, the visual biofeedback module can combine muscle activation data with corresponding video data to reveal the subject's movement at the moment when a certain EMG pattern was captured. This improved approach not only empowers therapists and movement professionals to analyze movement video together with synchronized muscle firing patterns, but also functions as a biofeedback instrument for the subject performing the functional movements. For example, the subject can highlight a frame that indicates the moment the muscle sending EMG data should have activated. With this visual feedback, the subject can determine whether the target muscle activated when it should have, and then use this information to improve the firing pattern in future functional movements. With practice, the subject can achieve a proper firing pattern within the functional movement, and relieve or prevent joint or muscle pain caused by faulty movements or muscle firing patterns.

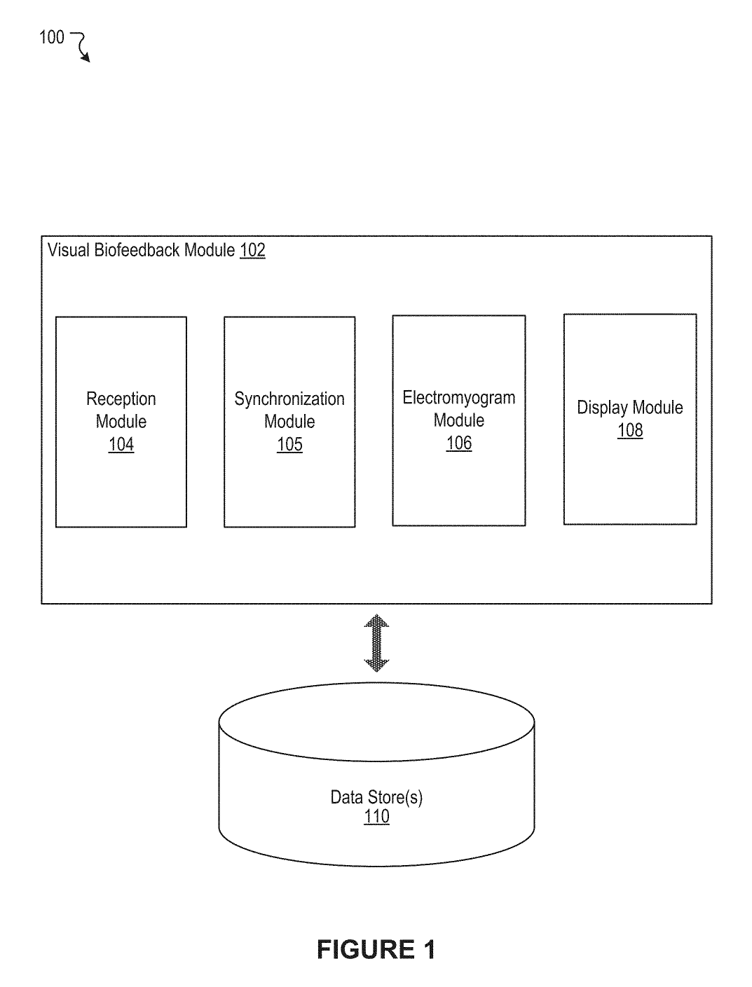

With reference now to FIG. 1 of the illustrative drawings, there is shown an example system 100 including an example visual biofeedback module 102 according to an embodiment of the present disclosure. The visual biofeedback module 102 can be configured to receive video data, and corresponding video timestamp data, of a subject performing functional movements; and receive EMG data, and corresponding EMG timestamp data, from an electromyograph attached to the subject performing the functional movements. The visual biofeedback module 102 can be configured to synchronize the received EMG data and the received video data using the received EMG timestamp data and the received video timestamp data; generate at least one electromyogram from the synchronized EMG data; and display the synchronized video data and the at least one electromyogram.

As shown in the example of FIG. 1, the visual biofeedback module 102 can include a reception module 104, a synchronization module 105, an electromyogram module 106, and a display module 108. In some instances, the example system 100 can include at least one data store 110. The components (e.g., modules, elements, etc.) shown in this figure and all figures in this application are exemplary only, and other implementations can include additional, fewer, integrated, or different components. Some components might not be shown so as to clarify relevant details. In various embodiments, one or more functionalities described in connection with the visual biofeedback module 102 can be implemented in any suitable combination.

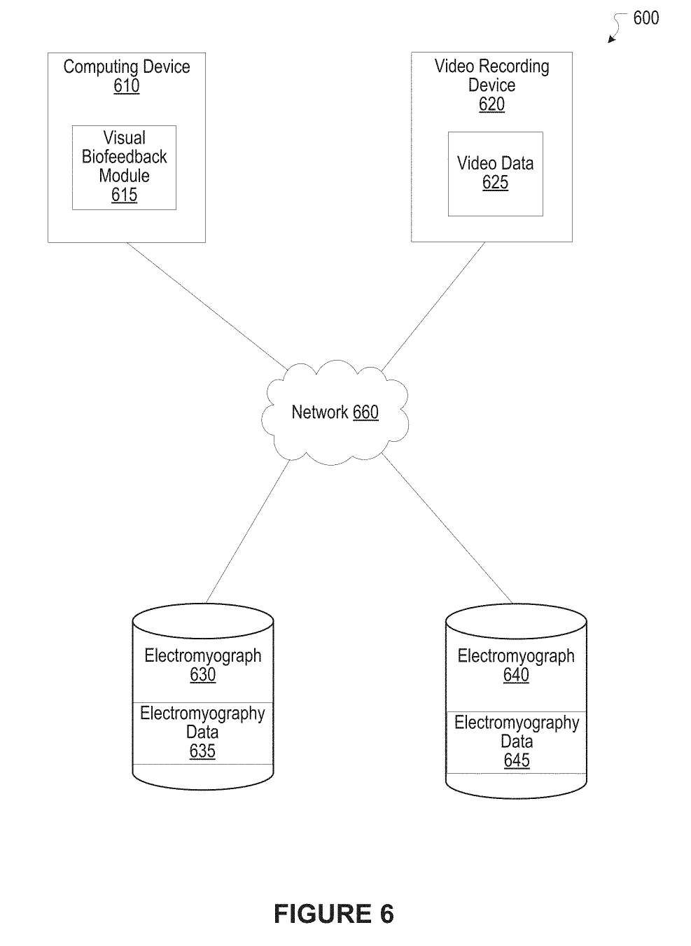

In some embodiments, the visual biofeedback module 102 (and any of the other modules described in this application) can be implemented, in part or in whole, as software, hardware, or any combination thereof. In some implementations, one or more functions, tasks, or operations of modules can be carried out or performed by software routines, software processes, hardware, or any combination thereof. In some cases, the visual biofeedback module 102 can be implemented, in part or in whole, as software running on one or more computing devices or systems, such as on a user or client computing device. For example, the visual biofeedback module 102, or at least a portion of it, can be implemented as or within an application (e.g., app), a program, a driver, or an applet, etc., running on a user computing device or a client computing system, such as the computing device 610 of FIG. 6. In another example, the visual biofeedback module 102, or at least a portion of it, can be implemented using one or more computing devices or systems that include one or more servers, such as network servers or cloud servers. It should be understood that there can be many variations or other possibilities.

The reception module 104 can be configured to receive video data, and corresponding timestamp data, as well as EMG data, and corresponding EMG timestamp data. In some embodiments, the video data and EMG data can be recorded as a subject performs functional movements. The video data can be video of the subject as the subject performs the functional movements, and the EMG data can represent electrical activity produced by at least one of the subject's muscles as the subject performs the functional movements. In some embodiments, the EMG data can be received from an electromyograph attached to the subject performing the functional movements. The reception module 104 is described in greater detail below.

The synchronization module 105 can be configured to synchronize the received EMG data and the received video data using the received EMG timestamp data and the received video timestamp data. In one embodiment, the synchronization module 105 can be configured to read the EMG timestamp data and the video timestamp data to identify overlapping times. The synchronization module 105 can then be configured to shift the EMG data, the video data, or both, so that the EMG timestamp data and the video timestamp data are aligned over a desired period. In one embodiment, the synchronization module 105 can facilitate synchronization by syncing a clock associated with the electromyograph with a clock associated with a device capable of collecting the video data. For example, in one embodiment, a hub, having a wireless connection to the electromyograph and the device capable of recording video, can update one or more of the clocks associated with the electromyograph and the device capable of recording video. In other embodiments, the device capable of recording video can directly update the clock associated with the electromyograph.

The electromyogram module 106 can be configured to generate at least one electromyogram from the synchronized EMG data. The electromyograph attached to the subject can detect electric potential signals generated by muscle cells when the cells are electrically or neurologically activated. The electromyogram module 106 can process these signals to produce the electromyogram. In one embodiment, the electromyogram can be a waveform that reflects the sum of the electrical contributions made by the active motor units in the vicinity of the electromyograph. In some embodiments, the electromyogram module 106 can analyze or process the synchronized EMG data before generating the at least one electromyogram. For example, the electromyogram module 106 can be configured to apply wavelet analysis, autoregressive time series models, artificial intelligence techniques, or high-order statistics to process the EMG data.

The display module 108 can be configured to display the synchronized video data and the at least one electromyogram. The display module 108 is described in greater detail below.

Furthermore, the visual biofeedback module 102 can be configured to communicate or operate with at least one data store 110, as shown in the example system 100. The data store 110 can be configured to store and maintain various types of data. In some embodiments, the data store 110 can store information that is used by the visual biofeedback module 102. For example, the data store 110 can store EMG data, EMG timestamp data, video data, and video timestamp data. It is contemplated that there can be many variations or other possibilities.

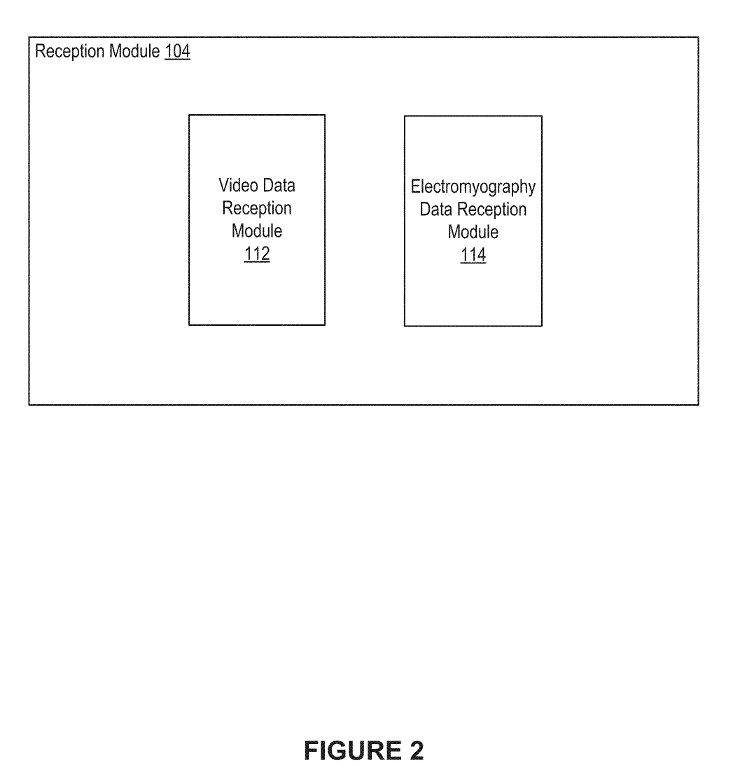

FIG. 2 illustrates a reception module 104 configured to receive video data, video timestamp data, EMG data, and EMG timestamp data, in accordance with one embodiment of the present disclosure. The reception module 104 can include a video data module 112 and an EMG data module 114.

The video data module 112 can be configured to receive video data, and corresponding video timestamp data, of a subject performing functional movements. In some embodiments, a video recording device, such as a personal computing device, mobile phone, or mobile tablet, can capture video data of the subject as the subject performs one or more functional movements. The video data module 112 can then receive the captured video data and corresponding video timestamp data.

The EMG data module 114 can be configured to receive EMG data, and corresponding EMG timestamp data, from an electromyograph attached to the subject performing the functional movements. In some embodiments, the EMG data module 114 can receive the EMG data and EMG timestamp data by wireless communication. In one embodiment, the electromyograph can comprise a first electromyograph. In another embodiment, the EMG data and EMG timestamp data received from the first electromyograph can comprise EMG data representing electrical activity produced by at least one proximal muscle. In a further embodiment the at least one proximal muscle can be selected from a group consisting of: transverse abdominis, multifidus, diaphragm, pelvic floor, gluteus medius, gluteus minimis, lower trapezius, suprasinatus, infraspinatus, teres minor, and subscapularis.

In one embodiment, the electromyograph can further comprise a second electromyograph. In another embodiment, the EMG data and EMG timestamp data received from the second electromyograph can comprise EMG data representing electrical activity produced by at least one distal muscle. In a further embodiment, the EMG data module 114 can be configured to receive EMG data and EMG timestamp data from both the first and second electromyographs. In an additional embodiment, the EMG data module 114 can be configured to aggregate the EMG data and the EMG timestamp data received from the first and second electromyograph.

In one embodiment, the at least one proximal muscle is the gluteus medius and the at least one distal muscle is the tensor fascia latae. In another embodiment, the at least one proximal muscle is the suprasinatus, infraspinatus, teres minor, or subscapularis, and the at least one distal muscle is the upper trapezius or deltoid. In a further embodiment, the at least one proximal muscle is the multifidus and the at least one distal muscle is the erector spinae. In an additional embodiment, the at least one proximal muscle is the gluteus maximus and the at least one distal muscle is the hamstring.

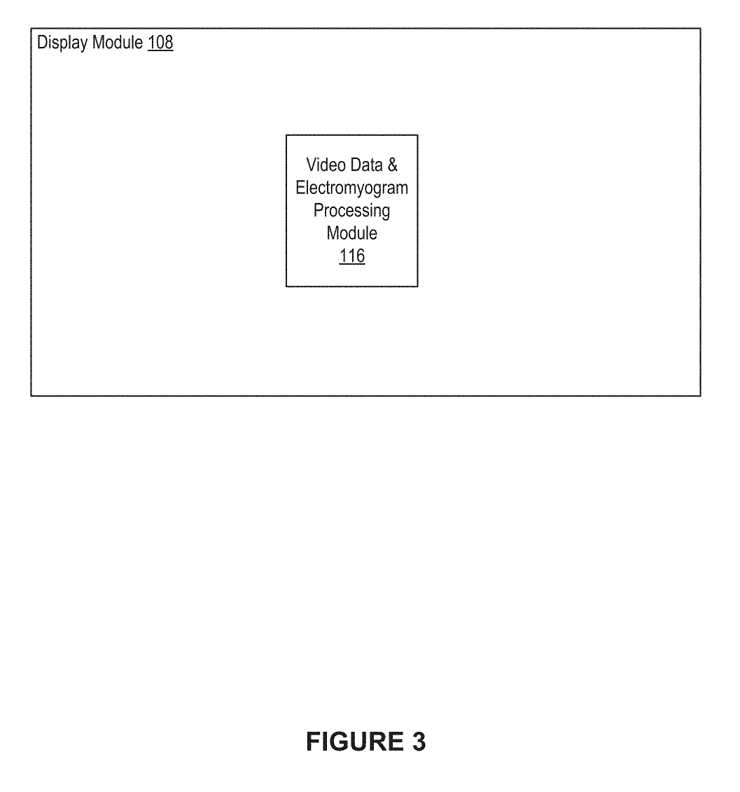

With reference now to FIG. 3, the display module 108 can include a video data and EMG processing module 116. In one embodiment, the video data and EMG processing module 116 can be configured to add the at least one electromyogram to the synchronized video data so that they can be displayed together. In another embodiment, the video data and EMG processing module 116 can be configured to overlay the at least one electromyogram to the synchronized video data so that they can be displayed together.

In one embodiment, the display module 108 can include an anatomy module (not shown), which can be configured to synchronize the at least one electromyogram with an anatomy illustration and display the synchronized anatomy illustration and the at least one electromyogram.

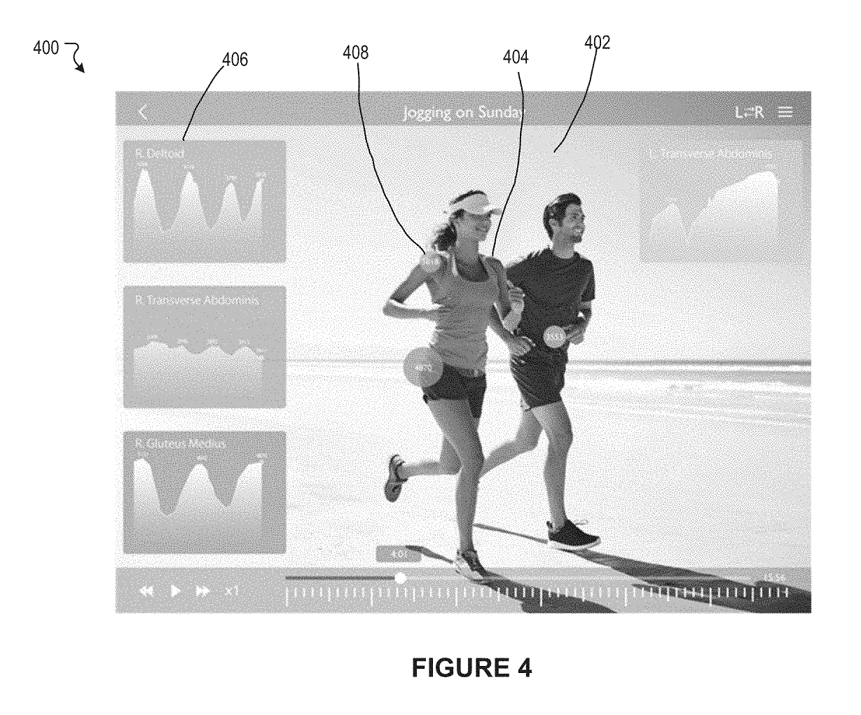

FIG. 4 illustrates an example display interface 400, according to an embodiment of the present disclosure. A user can view the display interface 400 to view video footage 402 of a subject 404 performing functional movements, along with at least one electromyogram 406 that has been synchronized with the video footage 402. In the example display interface 400, a video 402 of the subject 404 performing functional movements is shown. The subject 404 is wearing a first electromyograph 408 proximate the right deltoid muscle. EMG data from this first electromyograph 408 was recorded as the subject performed the functional movements shown in the video 402. The recorded EMG data and video data are then received by the visual biofeedback module 102, as described above. After the EMG data and video data are processed and synchronized, the electromyogram 406 is displayed alongside the synchronized video data 402.

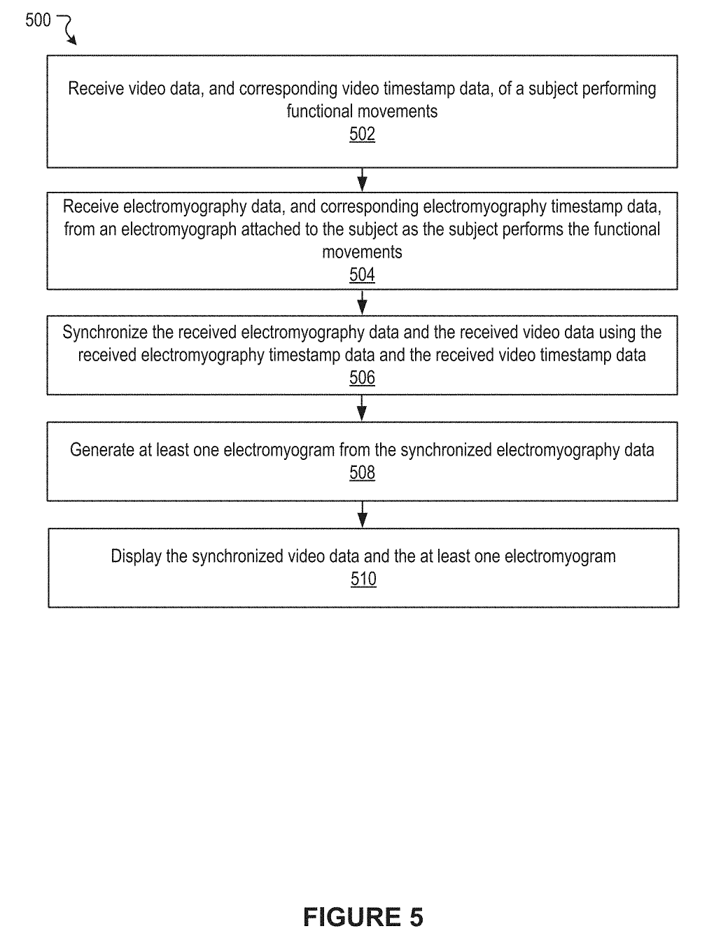

FIG. 5 illustrates an example method 500 associated with providing synchronized visual biofeedback data, according to an embodiment of the present disclosure. It should be appreciated that there can be additional, fewer, or alternative steps performed in similar or alternative orders, or in parallel, within the scope of various embodiments unless otherwise stated.

At block 502, the example method 500 can receive video data, and corresponding video timestamp data, of a subject performing functional movements. At block 504, the example method 500 can receive EMG data, and corresponding EMG timestamp data, from an electromyograph attached to the subject performing the functional movements At block 506, the example method 500 can synchronize the received EMG data and the received video data using the received EMG timestamp data and the received video timestamp data. At block 508 the example method 500 can generate at least one electromyogram from the synchronized EMG data. At block 510 the example method 500 can display the synchronized video data and the at least one electromyogram.

A subject might use an embodiment of this method, for example, to improve the functional performance of the transverse abdominis (TA) and mitigate back pain caused by lumbar spine instability. The TA is a proximal muscle. It is located in the deepest layer of the abdominal wall and functions to stabilize a subject's truck in almost all movement. A weakness or lack of control of the TA can contribute to lumbar spine instability and low back pain.

Traditionally, exercises such as the abdominal brace can be used to strengthen the TA and improve lumbar stability. However, many subjects are unable to eliminate back pain even after strengthening the TA with abdominal brace exercises. For these subjects, strength training, by itself, does not address an underlying issue with the TA muscle's motor-firing timing or muscle-activating precision. For example, a subject might have a strong TA muscle when she performs exercises like the abdominal brace; but be unable to activate the TA muscle properly when performing real-world functional movements, such as carrying a heavy object.

Using embodiments of the method 500 described above, the subject can practice activating the TA muscle during a functional movement such as twisting the trunk, lifting a golf club bag from the floor, and turning to walk in the opposite direction. In one embodiment, the EMG data detects the TA and the overlapping muscle activations while a mobile device records a video of the entire sequence of functional movements (e.g., twisting the trunk, bending over to lift the heavy object, grabbing the object, restoring the trunk, and turning to the opposite direction). The video and EMG data are synchronized using the timestamp data, and a video having a synchronized electromyogram is displayed. The synchronized electromyogram can allow a clinician to analyze whether the TA muscle was activated before the subject exerted force on the spine. If the TA muscle is activated after the subject lifts the object, the object will exert a shear force (which will not be not adequately countered by the core muscles) and displace the spinal column. By repeating this exercise and receiving visual feedback about whether the TA muscle was activated in the correct manner, the subject will eventually improve control of the TA muscle, even in fast and complex functional movements. By internalizing these controls over the TA muscle and other core muscles, the subject would eventually stabilize the spine and mitigate the low back pain.

A subject might also use embodiments of this method, for example, to improve the functional performance of the multifidus and mitigate back pain caused by lumbar spine instability. The multifidus is a proximal muscle. It is located between different segments of the vertebral column and attaches the transverse process and the spinal process. Activating the multifidus along one side causes rotation of the trunk and activating the multifidus along both sides stabilizes the spine. The multifidus functions to stabilize a subject's truck in almost all movement. A weakness or lack of control of the multifidus can contribute to lumbar spine instability and low back pain.

Traditionally, exercises such as the bird dog can be used to strengthen the multifidus and improve lumbar stability. However, many subjects are unable to eliminate back pain even after strengthening the multifidus with bird dog exercises. For these subjects, strength training, by itself, does not address an underlying issue with the multifidus muscle's motor-firing timing or muscle-activating precision. For example, a subject might have a strong multifidus muscle when he performs exercises like the bird dog; but the subject might be unable to activate the multifidus muscle properly when performing real-world functional movements, such as carrying a heavy object or twisting the trunk during a movement.

Using embodiments of the method 500 described above, the subject can practice activating the multifidus muscle during a functional movement such as twisting the trunk, lifting a golf club bag from the floor, and turning to walk in the opposite direction. In one embodiment, the EMG data detects the multifidus and the overlapping muscle activations while a mobile device records a video of the entire sequence of functional movements (e.g., twisting the trunk, bending over to lift the heavy object, grabbing the object, restoring the trunk, and turning to the opposite direction). The video and EMG data are synchronized using the timestamp data, and a video having a synchronized electromyogram is displayed. The synchronized electromyogram can allow a clinician to analyze whether the multifidus muscle was activated before the subject exerted force on the spine. If the multifidus muscle is activated after the subject lifts the object, the object will exert a torque, which will not be not countered by the core muscles, and displace the spinal column. By repeating this exercise and receiving visual feedback about whether the multifidus muscle was activated in the correct manner, the subject would eventually improve control of the multifidus muscle, even in fast and complex functional movements. By internalizing these controls over the multifidus muscle and other core muscles, the subject would eventually stabilize the spine and mitigate the low back pain.

In some embodiments of method 500, the received EMG data can comprise EMG data received from a first electromyograph and EMG data received from a second electromyograph. In one embodiment, the EMG data received from the first electromyograph can comprise EMG data representing electrical activity produced by at least one proximal muscle, and the EMG data received from the second electromyograph can comprise EMG data representing electrical activity produced by at least one distal muscle.

For example, the EMG data received from the first electromyograph can comprise EMG data representing electrical activity produced by the multifidus, and the EMG data received from the second electromyograph can comprise EMG data representing electrical activity produced by the erector spinae. Back stability involves strengthening back extensors, including the multifidus and erector spinae. However, an overactive erector spinae can cause lumbar lordosis and low back pain, while a stronger multifidus can increase stability and improve ability to resist shear and rotational load on the spine. Accordingly, embodiments of the method 500 can be used to adjust exercises so as to increase multifidus involvement and reduce erector spinae involvement.

In another example, the EMG data received from the first electromyograph can comprise EMG data representing electrical activity produced by the gluteus medius, and the EMG data received from the second electromyograph can comprise EMG data representing electrical activity produced by the tensor fascia latae. In normal gait pattern, the hip abductors (including gluteus medius, gluteus minimis, and tensor fascia latae) hold the pelvis level and prevent pelvic drop, or Trendelenburg Sign. Weak hip abductors, particularly the gluteus medius, can lead to Trendelenburg Sign. Walking or running with Trendelenburg Sign can result in uneven force distribution to the knee, which can cause pain in the knee. If the hip abductors are improperly strengthened, the tensor fascia latae can become pathologically tight, which can cause excessive pressure on the iliotibial bursa. Eventually, the bursa can become inflamed, which can cause a pain in the knee known as ITB Syndrome. Accordingly, embodiments of the method 500 can be used to strengthen the gluteus medius without over-activating the tensor fascia latae.

In another example, the EMG data received from the first electromyograph can comprise EMG data representing electrical activity produced by the rotator cuffs (e.g., suprasinatus, infraspinatus, teres minor, and subscapularis), and the EMG data received from the second electromyograph can comprise EMG data representing electrical activity produced by the upper trapezius or deltoid. The scapula is not attached directly to the spine and is positioned by the pulling of surrounding muscles. Proper biomechanics of full shoulder flexion or abduction involves the proper activation of the rotator cuffs, which connect the scapula and the humerus across the glenohumeral joint, as well as the activation of the serratus anterior, which tips the inferior angle of the scapula laterally and upward. If the rotator cuffs or the serratus anterior muscles become excessively weak, or the upper trapezius becomes more dominant in relationship to the lower trapezius, the upper trapezius will compensate to direct shoulder flexion or abduction. The upper trapezius is a large muscle that can pull the scapula away from its proper position and cause injury to the smaller rotator cuffs. Accordingly, embodiments of the method 500 can be used to adjust exercises so as to increase involvement of the rotator cuffs and reduce upper trapezius or deltoid involvement.

In another example, the EMG data received from the first electromyograph can comprise EMG data representing electrical activity produced by the gluteus maximus, and the EMG data received from the second electromyograph can comprise EMG data representing electrical activity produced by the hamstrings (e.g., semimembranosus, semitendinosus, and biceps femoris). During running, the gluteus maximus and the hamstrings are involved in driving the leg backward to propel the body. Strengthening the gluteus maximus without overacting the hamstrings can improve running and reduce running injuries. Accordingly, embodiments of the method 500 can be used to adjust exercises so as to increase involvement of the gluteus maximus and reduce involvement of the hamstrings.

### Biofeedback System—Example Implementation

FIG. 6 illustrates a network diagram of an example system 600 that can be used in various scenarios, according to an embodiment of the present disclosure. The system includes a computing device 610, a visual biofeedback module 615, a video recording device 620, video data 625, a first electromyograph 630 having EMG data 635, a second electromyograph 640 having EMG data 645, and a network.

In one embodiment, the computing device discussed in connection with the embodiments described above can be implemented as the computing device 610. For purposes of illustration, the embodiment of the system 600, shown by FIG. 6, includes a single computing device 610, a single video recording device, and two electromyograph sensors 630, 640. However, in other embodiments, the system 600 can include more computing devices, video recording databases, or electromyograph sensors. Furthermore, in some embodiments, the video recording device can be part of the computing device. For example, in one embodiment, the computing device and the video recording device can be embodied in a personal computing device such as an iPhone or an iPad.

The computing device 610 can comprise one or more computing devices that can receive input from a user and transmit and receive data via the network 660. Furthermore, while the visual biofeedback module 615 is shown as being directly connected to the computing device 610, it should be understood that it can be connected to the network 660 and can be connected to the center computing device 610 via the network 660. In one embodiment, the computing device 610 is a conventional computer system executing, for example, a Microsoft Windows compatible operating system (OS), Apple OS X, or a Linux distribution. In another embodiment, the computing device 610 can be a device having computer functionality, such as a smart-phone, a tablet, a personal digital assistant (PDA), a mobile telephone, etc. The computing device 610 can be configured to communicate via the network 660. The computing device 610 can execute applications, for example, a browser application that allows a user of the computing device 610 to interact with the video data 625 and the EMG data 635, 645.

The video recording device 620 can comprise one or more video recording devices that can record video. In some embodiments, video recording device 620 can transmit the recorded video via the network 660. Furthermore, while the video data 625 is shown as being directly connected to the video recording device 620, it should be understood that it can be connected to the network 660 and can be connected to the video recording device 620 via the network 660. In one embodiment, the video recording device 620 is a conventional video camera or movie camera. In another embodiment, the video recording device 620 can be a device having camera functionality, such as a smart-phone, a tablet, a personal digital assistant (PDA), a mobile telephone, etc. The computing device 620 can be configured to communicate via the network 660.

The first electromyograph 630 and the second electromyograph 640 can each comprise one or more sensors configured to record EMG data. In one embodiment, the first electromyograph 630 and the second electromyograph 640 can each comprise a surface electromyograph. In some embodiments, each of the first and second electromyographs 630, 640 can comprise a plurality of electrodes. Each of the first and second electromyographs 630, 640 can be configured to communicate via the network 660. In one embodiment, each of the first and second electromyographs 630, 640 can be configured to transmit the EMG data to the computing device 610 wirelessly. In another embodiment, each of the first and second electromyographs 630, 640 can be configured to transmit the EMG data to a hub (not shown), which can be configured to transmit the EMG data to the computing device 610. In some embodiments, the hub can be configured to aggregate and synchronize the EMG data, as described above, before transmitting the EMG data to the computing device 610.

The network 660 can comprise any combination of local area or wide area networks, using wired or wireless communication systems. In one embodiment, the network 660 uses standard communications technologies and protocols. Thus, the network 660 can include links using technologies such as Ethernet, 802.11, worldwide interoperability for microwave access (WiMAX), 3G, 4G, CDMA, GSM, LTE, digital subscriber line (DSL), etc. In addition, the network 660 can include links using wireless personal area networks such as Bluetooth or Bluetooth LE. Similarly, the networking protocols used on the network 660 can include multiprotocol label switching (MPLS), transmission control protocol/Internet protocol (TCP/IP), User Datagram Protocol (UDP), hypertext transport protocol (HTTP), simple mail transfer protocol (SMTP), file transfer protocol (FTP), and the like. The data exchanged over the network 660 can be represented using technologies or formats including hypertext markup language (HTML) and extensible markup language (XML). In addition, all or some links can be encrypted using conventional encryption technologies such as secure sockets layer (SSL), transport layer security (TLS), and Internet Protocol security (IPsec).

### Hardware Implementation

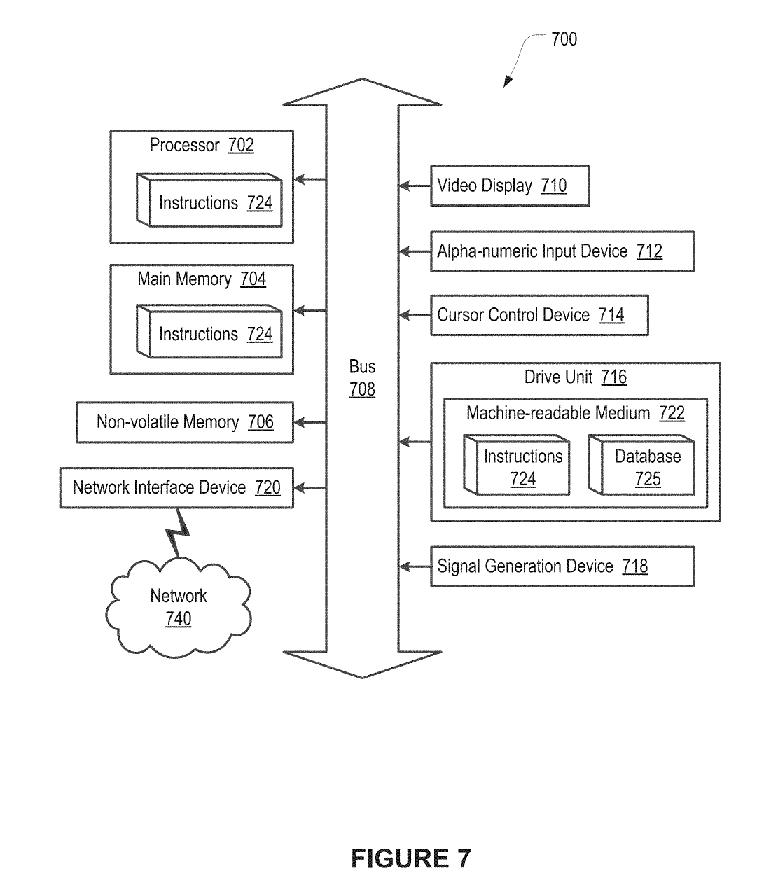

FIG. 7 is a diagrammatic representation of an embodiment of a machine 700, within which a set of instructions for causing the machine to perform one or more of the described embodiments can be executed. The machine can be connected (e.g., networked) to other machines. In a networked deployment, the machine can operate in the capacity of a server or a client machine in a client-server network environment, or as a peer machine in a peer-to-peer (or distributed) network environment. In one embodiment, the machine communicates with the server to facilitate operations of the server or to access the operations of the server.

The machine 700 includes a processor 702 (e.g., a central processing unit (CPU), a graphics processing unit (GPU), or both), a main memory 704, and a nonvolatile memory 706 (e.g., volatile RAM and non-volatile RAM), which communicate with each other via a bus 708. In some embodiments, the machine 700 can be a desktop computer, a laptop computer, personal digital assistant (PDA), a mobile phone, or a tablet, for example. In one embodiment, the machine 700 also includes a video display 710, an alphanumeric input device 712 (e.g., a keyboard), a cursor control device 714 (e.g., a mouse), a drive unit 716, a signal generation device 718 (e.g., a speaker) and a network interface device 720.

In one embodiment, the video display 710 includes a touch sensitive screen for user input. In one embodiment, the touch sensitive screen is used instead of a keyboard and mouse. The disk drive unit 716 includes a machine-readable medium 722 on which is stored one or more sets of instructions 724 (e.g., software) embodying any one or more of the described methodologies or functions. The instructions 724 can also reside, completely or at least partially, within the main memory 704 or within the processor 702 during execution thereof by the computer system 700. The instructions 724 can further be transmitted or received over a network 740 via the network interface device 720. In some embodiments, the machine-readable medium 722 also includes a database 725.

Volatile RAM can be implemented as dynamic RAM (DRAM), which requires power continually in order to refresh or maintain the data in the memory. Non-volatile memory is typically a magnetic hard drive, a magnetic optical drive, an optical drive (e.g., a DVD RAM), or other type of memory system that maintains data even after power is removed from the system. The non-volatile memory can also be a random access memory. The non-volatile memory can be a local device coupled directly to the rest of the components in the data processing system. A non-volatile memory that is remote from the system, such as a network storage device coupled to any of the described computer systems through a network interface such as a modem or Ethernet interface, can also be used.

While the machine-readable medium 722 is shown in an exemplary embodiment to be a single medium, the term “machine-readable medium” should be taken to include a single medium or multiple media (e.g., a centralized or distributed database, or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure. The term “machine-readable medium” shall accordingly be taken to include, but not be limited to, solid-state memories, optical and magnetic media, and carrier wave signals. The term “storage module” as used in this disclosure can be implemented using a machine-readable medium.

In general, routines executed to implement the embodiments of the invention can be implemented as part of an operating system or a specific application, component, program, object, module or sequence of instructions referred to as “programs” or “applications.” For example, one or more programs or applications can be used to execute any or all of the described functionality, techniques, and processes. The programs or applications typically comprise one or more instructions, set at various times in various memory and storage devices in the machine, that, when read and executed by one or more processors, cause the machine to perform operations to execute elements involving the various aspects of the described embodiments.

The executable routines and data can be stored in various places, including, for example, ROM, volatile RAM, non-volatile memory, or cache. Portions of these routines or data can be stored in any one of these storage devices. Further, the routines and data can be obtained from centralized servers or peer-to-peer networks. Different portions of the routines and data can be obtained from different centralized servers or peer-to-peer networks at different times and in different communication sessions, or in a same communication session. The routines and data can be obtained in entirety prior to the execution of the applications. Alternatively, portions of the routines and data can be obtained dynamically, just in time, when needed for execution. Thus, it is not required that the routines and data be on a machine-readable medium in entirety at a particular instance of time.

It should be appreciated from the foregoing description that the present invention provides an improved visual biofeedback method that not only provides insight into muscle activation patterns of target muscles during complex, functional movement, but that also provides synchronized visual feedback on the timing of the muscle activation relative to the functional movement.

For purposes of explanation, numerous specific details are set forth to provide a thorough understanding of the description. It will be apparent, however, to one skilled in the art, that embodiments of the disclosure can be practiced without these specific details. In some instances, modules, structures, processes, features, and devices are shown in block diagram form to avoid obscuring the description. In other instances, functional block diagrams and flow diagrams are shown to represent data and logic flows. The components of block diagrams and flow diagrams (e.g., modules, engines, blocks, structures, devices, features, etc.) can be variously combined, separated, removed, reordered, and replaced in a manner other than as expressly described and depicted.

Specific methods, devices, and materials are described, although any methods and materials similar or equivalent to those described can be used in the practice or testing of the present embodiment. Unless defined otherwise, all technical and scientific terms used in this disclosure have the same meanings as commonly understood by one of ordinary skill in the art to which this embodiment belongs.

The terms “a,” “an,” and “at least one” encompass one or more of the specified element. That is, if two of a particular element are present, one of these elements is also present and thus “an” element is present. The terms “a plurality of” and “plural” mean two or more of the specified element.

The term “or” used between the last two of a list of elements means any one or more of the listed elements. For example, the phrase “A, B, or C” means “A, B, and/or C,” which means “A,” “B,” “C,” “A and B,” “A and C,” “B and C,” or “A, B, and C.”

Without further elaboration, it is believed that one skilled in the art, using the proceeding description, can make and use the present invention to the fullest extent. The invention has been described in detail with reference only to the presently preferred embodiments. Persons skilled in the art will appreciate that various modifications can be made without departing from the invention. Accordingly, the invention is defined only by the following claims.

1. A computer-implemented method, implemented by a computing system, the method comprising:
receiving video data, and corresponding video timestamp data, of a subject performing functional movements;
receiving electromyography data, and corresponding electromyography timestamp data, from an electromyograph attached to the subject performing the functional movements;
synchronizing the received electromyography data and the received video data using the received electromyography timestamp data and the received video timestamp data;
generating at least one electromyogram from the synchronized electromyography data; and
displaying the synchronized video data and the at least one electromyogram.

2. The computer-implemented method of claim 1, wherein the electromyograph is a surface electromyograph.

3. The computer-implemented method of claim 2, wherein the electromyograph comprises a first electromyograph, wherein the electromyography data and electromyography timestamp data received from the first electromyograph comprises electromyography data representing electrical activity produced by at least one proximal muscle selected from a group consisting of: transverse abdominis, multifidus, diaphragm, pelvic floor, gluteus medius, gluteus minimis, lower trapezius, suprasinatus, infraspinatus, teres minor, and subscapularis.

4. The computer-implemented method of claim 3, wherein the electromyograph further comprises a second electromyograph, wherein the electromyography data and electromyography timestamp data received from the second electromyograph comprises electromyography data representing electrical activity produced by at least one distal muscle.

5. The computer-implemented method of claim 4, wherein receiving the electromyography data and the corresponding electromyography timestamp data comprises receiving electromyography data and electromyography timestamp data from both the first and second electromyographs.

6. The computer-implemented method of claim 5, wherein receiving the electromyography data and the corresponding electromyography timestamp data further comprises aggregating the electromyography data and the electromyography timestamp data received from the first and second electromyographs.

7. The computer-implemented method of claim 6, wherein each of the first and second electromyographs comprises a plurality of electrodes.

8. The computer-implemented method of claim 7, wherein displaying the synchronized video data and the at least one electromyogram comprises adding the at least one electromyogram to the synchronized video data.

9. The computer-implemented method of claim 7, wherein the electromyography data, the electromyography timestamp, the video data, and the video timestamp data are received by wireless communication.

10. The computer-implemented method of claim 7, further comprising synchronizing the at least one electromyogram with an anatomy illustration; and displaying the synchronized anatomy animation and the at least one electromyogram.

11. A system comprising:
at least one processor; and
a memory storing instructions that, when executed by the at least one processor, cause the system to perform a method comprising:
receiving video data, and corresponding video timestamp data, of a subject performing functional movements;
receiving electromyography data, and corresponding electromyography timestamp data, from an electromyograph attached to the subject performing the functional movements;
synchronizing the received electromyography data and the received video data using the received electromyography timestamp data and the received video timestamp data;
generating at least one electromyogram from the synchronized electromyography data; and
displaying the synchronized video data and the at least one electromyogram.

12. The system of claim 11, wherein the electromyograph is a surface electromyograph.

13. The system of claim 12, wherein the electromyograph comprises a first electromyograph, wherein the electromyography data and electromyography timestamp data received from the first electromyograph comprises electromyography data representing electrical activity produced by at least one proximal muscle.

14. The system of claim 13, wherein the electromyograph further comprises a second electromyograph, wherein the electromyography data and electromyography timestamp data received from the second electromyograph comprises electromyography data representing electrical activity produced by at least one distal muscle.

15. The system of claim 14, wherein receiving the electromyography data and the corresponding electromyography timestamp data comprises receiving electromyography data and electromyography timestamp data from both the first and second electromyographs and aggregating the electromyography data and the electromyography timestamp data received from the first and second electromyographs.

16. A method of strength training, the method comprising:
activating a target proximal muscle and a distal muscle during a functional movement;
receiving visual biofeedback, wherein the visual biofeedback comprises video of the functional movement and at least one electromyogram; and
adjusting muscle activation timing of the target proximal muscle during the functional movement in response to the visual biofeedback;
wherein the at least one electromyogram is generated from electromyography data received from a first surface electromyograph and electromyography data received from a second surface electromyograph;
wherein the electromyography data received from the first surface electromyograph comprises electromyography data representing electrical activity produced by the target proximal muscle;
wherein the electromyography data received from the second surface electromyograph comprises electromyography data representing electrical activity produced by the distal muscle; and
wherein the video of the functional movement is synchronized with the at least one electromyogram by synchronizing video timestamp data and electromyography timestamp data from the first and second surface electromyographs.

17. The method of claim 16, wherein the target proximal muscle is the gluteus medius and the distal muscle is the tensor fascia latae.

18. The method of claim 16, wherein the target proximal muscle is the suprasinatus, infraspinatus, teres minor, or subscapularis, and the distal muscle is the upper trapezius or deltoid.

19. The method of claim 16, wherein the target proximal muscle is the multifidus and the distal muscle is the erector spinae.

20. The method of claim 16, wherein the target proximal muscle is the gluteus maximus and the distal muscle is the hamstring.
